(19) United States
(12) Patent Application Publication
(10) Pub. No.: US 2015/0320964 A1
(43) Pub. Date: Nov. 12, 2015

(54) STERILE LIQUID DELIVERY SYSTEM AND METHOD

(71) Applicant: Black Cat Medical, LLC, Fishers, IN (US)

(72) Inventor: Michael F. Guzman, Fishers, IN (US)

(21) Appl. No.: 14/271,537

(22) Filed: May 7, 2014

(51) Int. Cl. A61M 19/00; A61B 8/00; A61M 5/19

(52) U.S. Cl. CPC A61M 19/00; A61M 5/19; A61B 8/00; A61M 2005/1787

(57) ABSTRACT

A medical device and method for providing liquid injectable into a patient from a first liquid container via and a needle is disclosed. One and two syringe devices are disclosed. Valving between the first syringe and the first liquid container allows liquid to be drawn from the first liquid container into the first syringe and to prevent liquid flow back toward the first liquid container. Methods of injecting sterile liquids and/or visualizing them are provided.

### BACKGROUND

The present invention relates to delivering liquids to patient via a needle. It has application in a variety of indications, including localized anesthesia.

Injecting liquids into patients generally is old. More recently, in the field of nerve blocks some anesthesiologists in the U.S. (and perhaps elsewhere) have injected a liposome suspension of bupivacaine (trademark Exparel®). An example is recounted by Ilfeld et. al stating in part “that deposition of a liposomal bupivacaine formulation adjacent to the femoral nerve results in a partial sensory and motor block of >24 hours for the highest doses examined.” Brian M. Ilfeld, et. a., Liposomal Bupivacaine as a Single-Injection Peripheral Nerve Block: a Dose-Response Study, Anesthia & Analgesia 117(5), 1248-56 (2013).

Bupivacaine in cases of accelerated absorption and/or intravascular injection can be cardiotoxic.

Often when used or mixed in ambient conditions it is best to handle bupivacaine under a ventilated hood. Also, the Joint Commission on Accreditation of Healthcare Organizations (JCAHO) standard MM.05.01.07 #1 which states: “A pharmacist, or pharmacy staff under the supervision of a pharmacist, compounds or admixes all compounded sterile preparations except in urgent situations in which a delay could harm the patient or when the product's stability is short.”

A publication, “Every Patient's Pain is Personal: A Frost & Sullivan White Paper Evaluating the Clinical Experiences of I-Flow's ON-Q vs. Pacira Pharmaceutical's EXPAREL®” indicates that “the adoption of EXPAREL® was rapid in 2013, but the aggregate clinical perspective is that ON-Q pumps deliver superior pain control over the full length of the post-operative period, and at a lower cost when looked at across the entire recovery period.”

Thus, there is a need for improvement.

### SUMMARY

The claims and only the claims set forth the invention. In certain examples, the present disclosure provides a medical device for providing liquid injectable into a patient from a first liquid container via and a needle. Valving between the first syringe and the first liquid container may allow liquid to be drawn from the first liquid container into the first syringe and to prevent liquid flow back toward the first liquid container. Optionally, a second syringe and other valving may be used. Methods of injecting sterile liquids and/or visualizing them are provided.

Further forms, objects, features, aspects, benefits, advantages, and embodiments of the present invention will become apparent from a detailed description and drawings provided herewith.

### BRIEF DESCRIPTION OF THE DRAWINGS

FIG. 1A is schematic diagram of the present device being used with patient P.

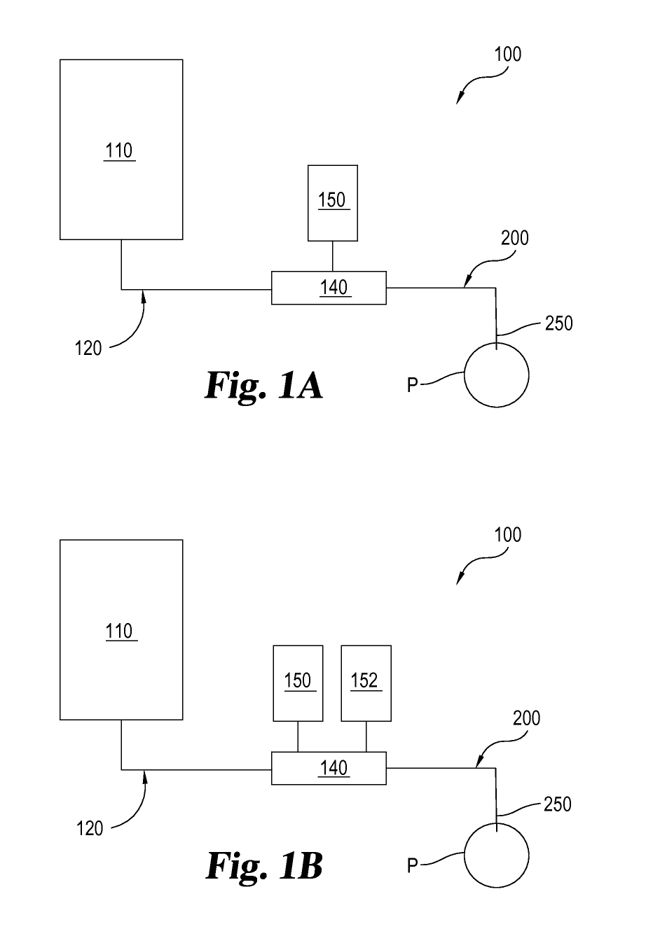

FIG. 1B is another schematic diagram of the present device being used with patient P.

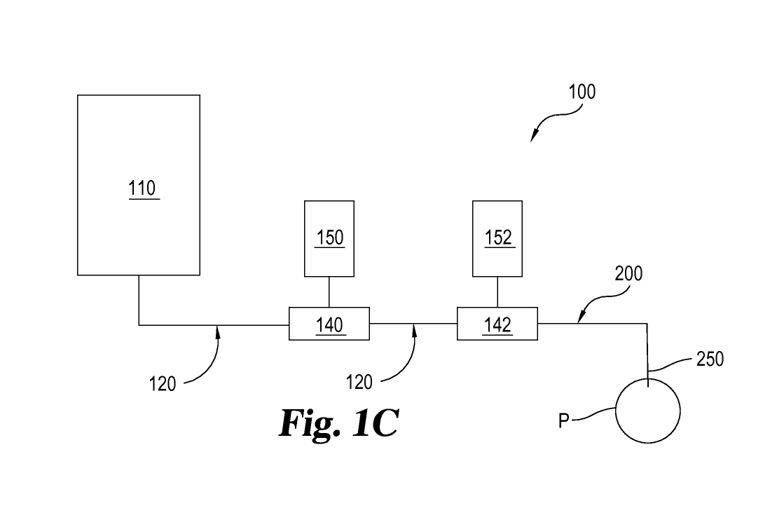

FIG. 1C is another schematic diagram of the present device being used with patient P.

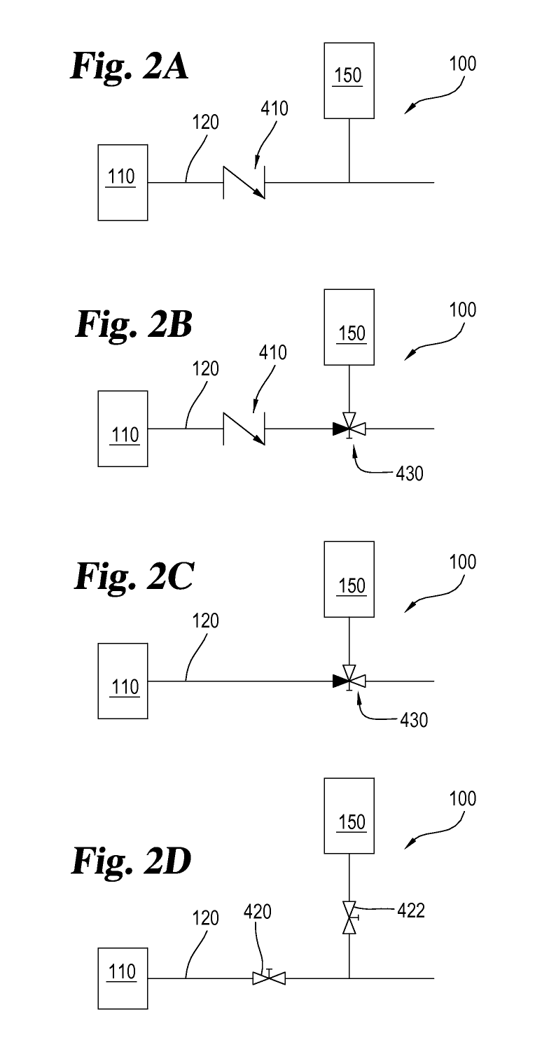

FIGS. 2A-2D are each partial schematics of examples of valving options.

FIG. 3 is a plan view of one example of the device and a wrist band.

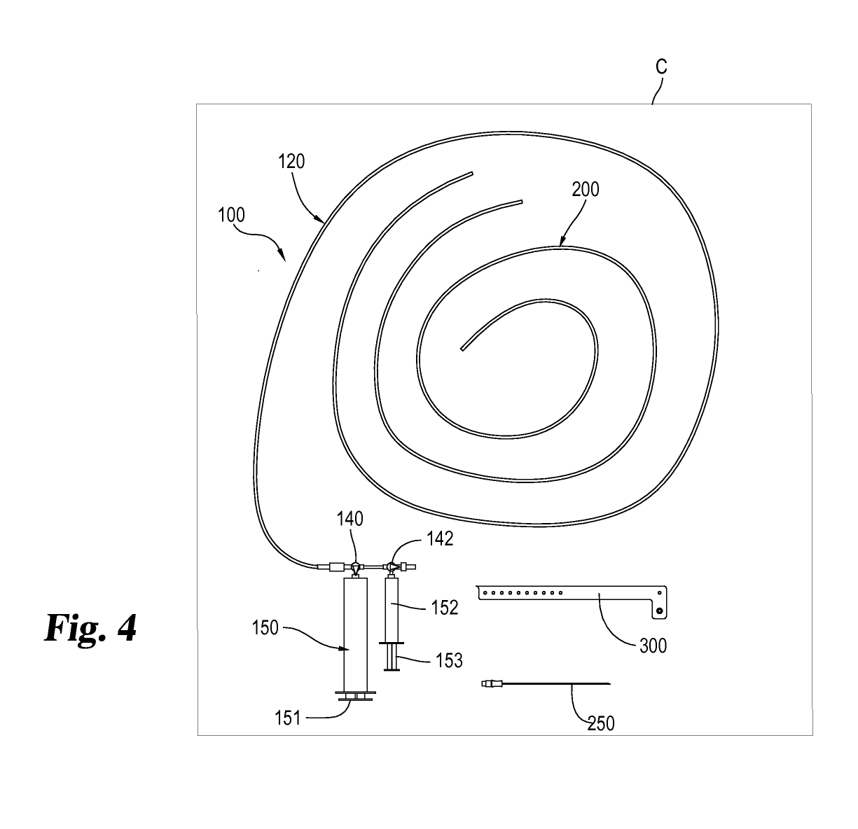

FIG. 4 is a plan view of one example of a kit with a container for parts of the device.

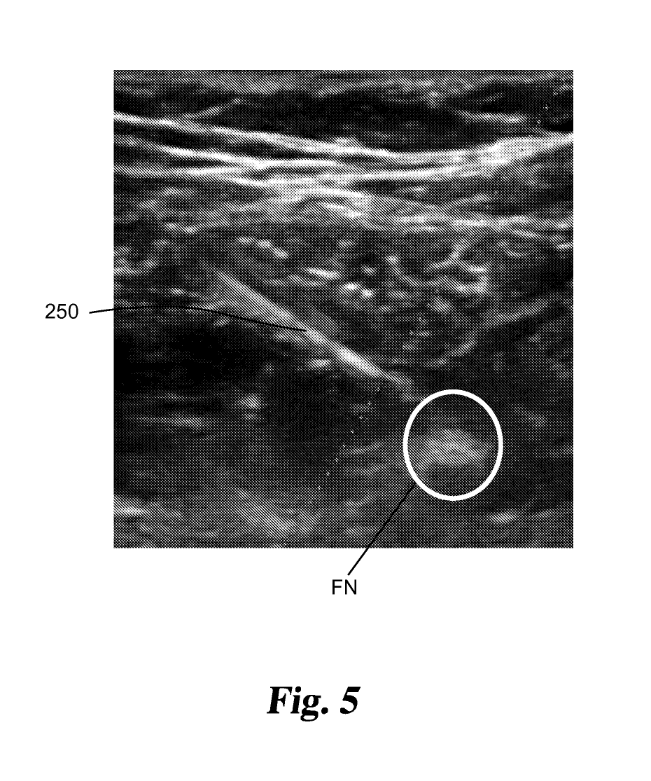

FIGS. 5-6 show in sequence ultrasonic images of one example of the device and method in use in a patient.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail; although it will be apparent to those skilled in the relevant art that some features that are not relevant to the present invention may not be shown for the sake of clarity.

In certain embodiments, the present disclosure provides a medical device 100 for providing liquid injectable into a patient from a first liquid container 110, 110a. In some forms, the medical device comprises at least: flexible sterile tubing 120, 120a, 200, 200a connectable at a proximal end to the first liquid container 110; an anesthesia needle 250 having a cannula at a distal end of the tubing 200, 200a; and a first syringe 150, 150a having a first plunger 151, 151a, the first syringe connected to the tubing between the needle and the first liquid container. The device also includes a first valving 140, 140a between the first syringe and the first liquid container to allow liquid to be drawn from the first liquid container into the first syringe upon retraction of the first plunger, and to prevent liquid flow back toward the first liquid container and, wherein liquid can be drawn from the first liquid container into the first syringe and then injected from the first syringe into the patient via the needle cannula.

Optionally but not necessarily the first liquid container contains a pharmaceutical anesthetic. Optionally but not necessarily the pharmaceutical anesthetic comprises bupivacaine, and/or ropivacaine, or may consist of and/or may consist essentially of bupivacaine or of ropivacaine. When such is the case or not, the pharmaceutical anesthetic may optionally comprise a liposome suspension or more broadly a polymer suspension.

Optionally but not necessarily the first liquid container has a volume of at least 40 milliliters of sterile preservative-free normal saline therein and contains the pharmaceutical anesthetic injected into the first container and diluted by the saline within the first container. However, other volumes of at least 50, 100 or 200 or more milliliters are options as well.

Optionally but not necessarily the medical device further comprises a second syringe 152, 152a having a second plunger 153, 153a, the second syringe connected to the tubing 120, 200, wherein a second liquid different from the liquid from the first liquid container can be injected from the second syringe into the patient via the needle cannula.

The device, optionally but not necessarily, may further comprise second valving 142, 142a between the second syringe and the tubing to allow liquid isolation of the second syringe. When such is the case, the first valving and the second valving may be directly connected to one another. In other forms, the first valving and the second valving are connected by a connector 210a. Connector 210a may be rigid or flexible, in some forms connector 210a comprises surgical tubing.

Optionally but not necessarily the second liquid in the second syringe has a lower ultrasound echogenicity than the liquid in the first container whereby injecting the second liquid locally increases ultrasound visibility. The second liquid may optionally but not necessarily comprise a local anesthetic.

The anesthesia needle may optionally but not necessarily comprises a local anesthesia needle at least 2.5 centimeters long and having at least one echogenic marker thereon.

The device, optionally but not necessarily may further comprise tubing (120 and 200 collectively) that may be at least 40, 50, or 60 centimeters long or longer.

Optionally but not necessarily the medical device may further comprise at least one marking to indicate the liquid contained therein. The marking may optionally but not necessarily be a color indicator. For example, the color indicator may be teal. This may correspond to conventional or other color coding for a given drug, such as but one example, teal for Exparel®.

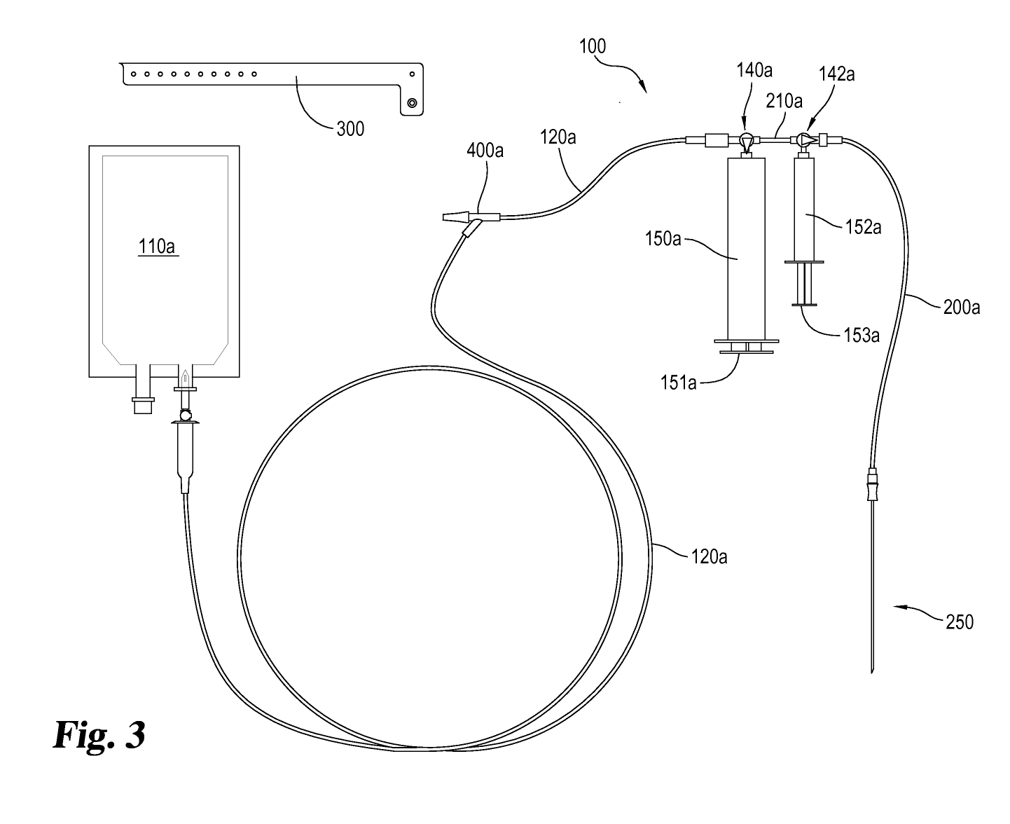

Valving may vary. Some examples include those shown schematically in FIGS. 2A, 2B 2C, 2D and/or combinations thereof or otherwise. Such valving may include, optionally but not necessarily the a check valve 410 along the length of the tubing to allow fluid flow in one direction (e.g. from the proximal end of the tubing toward the distal end) while preventing fluid backflow (e.g. towards the first liquid storage container). When used, a check valve may be present at any point along the tubing between the first liquid container and the needle. In some forms valving 140 and/or 142 comprise one or more stopcocks, including for example stopcocks 420, 422, 430. In some forms, a system of two-way stopcocks 420, 422 are used to direct fluid flow. In more preferred embodiments, one or more three-way stopcocks 430 are used to direct fluid flow. As discussed herein, any combination of tubing and valving may be employed to direct fluid flow as described herein.

Optionally but not necessarily the medical device further comprises a kit having an internal-sterile container C with at least: the tubing 120, 200, the first syringe 150, the first valving 140 and the needle 250 within the container. The kit may optionally but not necessarily contain a wrist band 300. The wrist band may, in some forms, comprise a color indicator. Optionally, other items may be included in the kit and/or container, include without limitation: a pharmaceutical anesthetic, a second syringe 152 (with or without a second liquid therein), a second valving 142, a protective sheath for the needle, instructions and/or otherwise.

In some forms, the device further comprises an optional port 400a along the length of the tubing 120, 120a, 200, 200a. In some forms the port may be adapted for further introduction of a liquid, for example to flush the tubing or administer additional material.

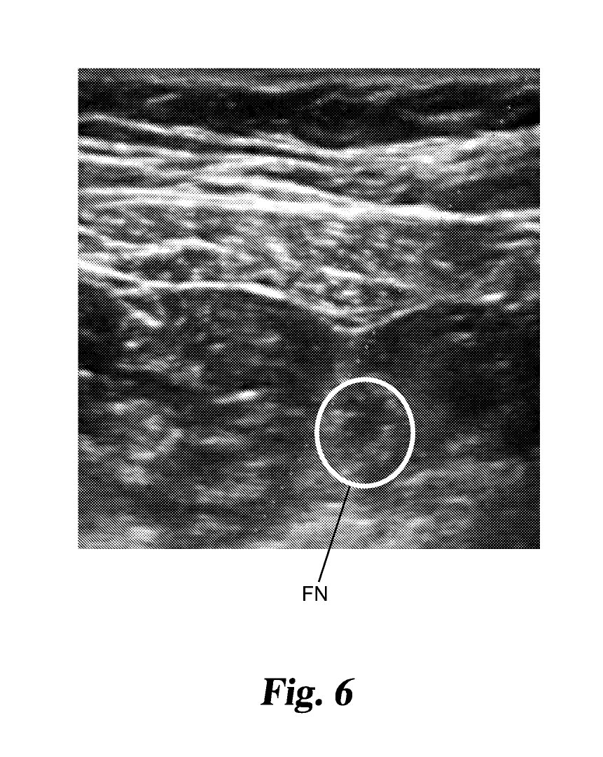

Exemplary methods for providing a liquid injectable into a patient from a first liquid container may be employed. The methods may comprise the acts of: connecting sterile tubing 120 at a proximal end to a first liquid container 110, the first liquid container containing a first liquid comprising a pharmaceutical anesthetic; retracting a first plunger 151 of a first syringe 150 to draw the first liquid into the first syringe via sterile flexible tubing 120 connecting the first syringe to the first liquid container; visualizing a local anesthesia needle in the patient via ultrasound imaging (see for example, FIGS. 5-6); while visualizing injecting the first liquid from the first syringe through the tubing and through a cannula of the needle at a distal end of the tubing into a local anesthesia site of the patient P; and, blocking the first liquid from flowing from the first syringe back toward the first liquid container with first valving 140.

As but one example, with reference to FIG. 5, shown is an image of an ultrasound needle 250 near a target femoral nerve FN, with normal saline solution surrounding the femoral nerve. With reference to FIG. 6, upon injection of a liposome injection (Exparel®) the landmarks, such as the needle and femoral nerve become difficult to visualize due in part to its high echogenicity.

The method optionally but not necessarily may include the acts of connecting a second fluid container 152 to the sterile tubing, the second fluid container containing a second liquid having lower echogenicity than the pharmaceutical anesthetic, injecting the second fluid through the tubing and through a cannula of the needle, and/or visualizing the second fluid via ultrasound imaging.

The method may optionally but not necessarily include the feature where the pharmaceutical anesthetic comprises a bupivacaine liposome suspension.

As used here (claims, specification, and other definitions) the following terms have the following meaning:

Articles and phases such as, “the”, “a”, “an”, “at least one”, and “a first”, “comprising”, “having” and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

The phrase “consisting essentially of” means specified materials and/or ingredient and/or steps and/or acts and/or other elements, and to those that do not materially affect the invention's basic and novel characteristics in the claim (as a transition) or the element or the group.

The phrase “consisting of” means literally to only include exactly what is stated, and excludes any step, act, material ingredient, or element not specified in the claim (as a transition) or the element or the group, but does not necessarily require the absence of trace amounts (less than 1% by weight) of impurities.

The term “and/or” is inclusive here, meaning “and” as well as “or”. For example, “P and/or Q” encompasses, P, Q, and P with Q; and, such “P and/or Q” may include other elements as well.

The term “means” and/or “means for” and/or “step” and/or “step for” here, if and when used in a claim, invokes 35 U.S.C. §112(f) means-plus-function for the recited function(s) and the corresponding structure(s) or “act(s)(including alternatives in the definitions or elsewhere in this disclosure) and equivalents thereto.

The term “anesthesia needle” as used herein has the meaning of a sterile cannulated needle suitable to deliver an anesthetic.

The term “blocking” as used herein has the meaning of preventing the flow of liquid.

The term “bupivacaine” as used herein refers to compositions containing a local anesthetic having as an active ingredient the chemical formula C18H28N2O and/or (RS)-1-butyl-N-(2,6-dimethylphenyl) piperidine-2-carboxamide and/or pharmaceutically acceptable salts, derivatives, metabolites, and/or prodrugs thereof.

The term “cannula” and “cannulated” as used herein has the meaning of or pertaining to a tube with at least one lumen for insertion into a patient. For example, a needle cannula may comprise a needle having an internal lumen or passage for delivery of a substance through the tube or passage.

The term “color indicator” as used herein has the meaning of a color used to communicate the identity or category of the liquid within the device. In some forms a color indicator is a solid color, in other forms the color indicator may be a pattern or plurality of colors. The color indictor may be present on any aspect of the present device(s), for example the liquid container, the flexible sterile tubing, the anesthesia needle, the first syringe, and/or the valving. The color indicator may also be present on an label configured to adhere to the liquid container. The color indicator may comprise any color used to communicate the identity of a substance, for example, a teal color may be used to indicate the presence of bupivacaine.

The term “connected” as used herein has the meaning that the connected aspects of the device are mechanically attached (directly and/or indirectly) and in the case of part(s) involved in fluid flow (for example valving, tubing, syringe), in fluid communication with each other.

The term “diluted” as used herein has the meaning of a solution having a reduced concentration of a reference chemical as compared to a starting liquid.

The term “diluted” as used herein has the meaning of a solution having a reduced concentration of a reference chemical as compared to a starting liquid.

The term “distal” as used herein has the meaning, relative to a reference, away from the doctor or other operator of the device; normally the opposite of “proximal”.

The term “drawn” as used herein has the meaning of moving liquid under suction and/or reduced pressure.

The term “echogenic marker” as used herein has the meaning of a marking having a different relative echogenicity than the surrounding structure.

The term “flexible sterile tubing” as used herein has the meaning of a flexible tubing, hose and/or catheter material with an outer wall surrounding a lumen, wherein the lumen is medically sterile. Normally, it is made of plastic. It may be transparent, opaque and/or translucent. It may comprise one or more segments, and typically where multiple segments are used they are connected by valves, interconnectors, luer lock structures, luer taper structures and/or otherwise.

The term “inject” and/or “injectable” as used herein refers and/or pertains to the capacity of a substance to be injected percutaneously into a patient.

The term “internal-sterile container” as used herein has the meaning of a sealed and container sterile on its inside.

The term “liposome suspension” as used herein refers to a pharmaceutical drug or agent (e.g. a pharmaceutical anesthetic) which has been encased, dissolved, emulsified or suspended in and/or within a liposome and/or other lipid suspendable (via colloidal action or otherwise) in a liquid. Normally, in this context it provides for delayed release of the pharmaceutical drug or agent over an extended time.

The term “liquid” as used herein has the meaning of a fluid other than gas.

The term “liquid container” as used herein has the meaning of a container capable of containing a liquid. It may be rigid and/or flexible. It may be made from plastic, glass, metal or otherwise.

The term “liquid isolation” as used herein has the meaning that the liquid isolated aspect of the device is not in fluid communication with another aspect of the device.

The term “local anesthesia needle” as used herein has the meaning of an anesthesia needle designed to deliver a local anesthetic. The anesthesia needle of the present disclosure may be any suitable size or length. In some forms anesthesia needles of the present disclosure have lengths of between to 1 cm to 26 cm, preferably 2.5 cm.

The term “marking” as used herein refers to a color, image, word, or words designed to readily communicate the identity or category of the liquid within the device. For example, the marking may be used to avoid use of contra-indicated drugs so as to avoid adverse reactions.

The term “medical device” as used herein has the meaning of a device useful for performing a medical procedure; for example, a device which would be used by a physician to aid with a diagnosis and/or therapeutic effect.

The term “nerve block” as used herein has the meaning of the act of injecting a nerve block agent and/or a nerve block agent, namely a local pharmaceutical anesthetic to temporarily stop or reduce localized pain and/or other sensation.

The term “patient” as used herein has the meaning a person or animal to be treated by a medical health care provider or by a veterinarian, respectively.

The term “pharmaceutical anesthetic” as used herein has the meaning of a substance (e.g a drug) that causes a loss of sensation. As used herein the term “pharmaceutical anesthetic” may refer to a general anesthetic and/or a local anesthetic. Such local anesthetics may include nerve block agent(s). In certain embodiments, a local anesthetic for use according to the present disclosure may comprise, for example, one or more of the following: procaine, amethocaine, cocaine, lidocaine, prilocaine, bupivacaine, levobupivacaine, ropivacaine, mepivacaine, and/or dibucaine.

The term “plunger” as used herein has the meaning of a portion of a plunger syringe adapted to be moved longitudinally to draw liquid into the syringe and/or force liquid from the syringe.

The term “polymer suspension” as used herein refers to a pharmaceutical drug or agent (e.g. a pharmaceutical anesthetic) which has been encased, dissolved, emulsified or suspended in and/or within a liposome and/or micelle and/or and/or polymer surfactant or tiny delivery compound suspendable (via colloidal action or otherwise) in a liquid. Normally, in this context it provides for delayed release of the pharmaceutical drug or agent over an extended time.

The term “preservative-free” as used herein has the meaning of substantially free of added preservatives.

The term “proximal” as used herein has the meaning, relative to a reference, towards the doctor or other operator of the device; normally the opposite of “distal”. The term “retraction” as used herein has the meaning of drawing or pulling back.

The term “ropivacaine” as used herein refers to compositions containing a local anesthetic having as an active ingredient the chemical formula C17H26N2O and/or (S)—N-(2,6-dimethylphenyl)-1-propylpiperidine-2-carboxamide and/or pharmaceutically acceptable salts, derivatives, metabolites, and/or prodrugs thereof.

The term “saline” as used herein has the meaning of a sterile solution of sodium chloride (NaCl) in water.

The term “sterile” as used herein has the meaning of substantially free from living germs or microorganisms.

The term “stopcock” as used herein has the meaning of a valve used to restrict or isolate the flow of liquid or gas through a tube (e.g. surgical tubing). For use in the present disclosure, a stopcock may comprise a 2-way, 3-way, or 4-way stop cock. As used herein a “two-way stopcock” is configured to allow liquid to flow therethrough from an input end to an output end, the two-way stopcock is further configured to prevent or block liquid flow in either direction. As used herein a “three-way stopcock” is configured to selectively allow liquid to flow in through any one of three ports while selectively allowing liquid outflow through one of the remaining two ports, while selectively restricting flow through the third port. A three-way stopcock may also be configured to allow fluid flow through each of the three ports.

The term “syringe” as used herein has the meaning of a device comprising an internal, sterile chamber that is collapsible to create pressure in a fluid within the chamber to force the fluid out of the chamber via an outlet. It may include a bulb syringe, a plunger syringe, a duel plunger syringe and/or otherwise. A syringe may be made of any suitable materials, for example, plastic, rubber or other elastomeric material, glass, and/or metal.

The term “ultrasound echogenicity” as used herein has the meaning of the propensity of a material (e.g. a liquid) to return an ultrasound echo. For example, if A has a higher ultrasound echogenicity than B, A will reflect more ultrasound sound waves (at detected frequenc(ies)) than B as appearing on ultrasound equipment.

The term “valving” as used herein has the meaning of device(s) and/or part(s) that regulate, direct or control the flow of a fluid by opening, closing, or partially obstructing various passageways. This may include, without limitation, 2-way valves, 3-way valves, check valves and/or otherwise.

The term “visualizing” as used herein has the meaning of to observe visual image(s), usually through the use of specialized equipment such as an ultrasound imaging device and/or monitor.

The invention may include any one or more articles or devices made by any of the claimed methods and/or may by different methods but with a claimed composition.

The language used in the claims and the written description and in the above definitions is to only have its plain and ordinary meaning, except for terms explicitly defined above. Such plain and ordinary meaning is defined here as inclusive of all consistent dictionary definitions from the most recently published (on the filing date of this document) general purpose Webster's dictionaries and Random House dictionaries.

While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A medical device for providing liquid injectable into a patient from a first liquid container, comprising:
flexible sterile tubing connectable at a proximal end to the first liquid container;
an anesthesia needle having a cannula at a distal end of said tubing;
a first syringe having a first plunger, said first syringe connected to said tubing between said needle and the first liquid container;
first valving between said first syringe and the first liquid container to allow liquid to be drawn from the first liquid container into said first syringe upon retraction of said first plunger, and to prevent liquid flow back toward the first liquid container; and,
wherein liquid can be drawn from the first liquid container into said first syringe and then injected from said first syringe into the patient via said needle cannula.

2. The device of claim 1 wherein the first liquid container contains a pharmaceutical anesthetic.

3. The device of claim 2 wherein the pharmaceutical anesthetic comprises bupivacaine.

4. The device of claim 2 wherein the pharmaceutical anesthetic comprises ropivacaine.

5. The device of claim 2 wherein the pharmaceutical anesthetic comprises a polymer suspension.

6. The device of claim 2 wherein the pharmaceutical anesthetic consists essentially of a bupivacaine liposome suspension.

7. The device of claim 2 wherein the pharmaceutical anesthetic consists essentially of a ropivacaine liposome suspension.

8. The device of claim 2 wherein the first liquid container has a volume of at least 40 milliliters of sterile preservative-free normal saline therein and contains said pharmaceutical anesthetic injected into said first container and diluted by said saline within said first container.

9. The device of claim 1 and further comprising a second syringe having a second plunger, said second syringe connected to said tubing, wherein a second liquid different from the liquid from the first liquid container can be injected from said second syringe into the patient via said needle cannula.

10. The device of claim 9 and further comprising second valving between said second syringe and said tubing to allow liquid isolation of said second syringe.

11. The device of claim 9 wherein the pharmaceutical anesthetic consists essentially of a bupivacaine liposome suspension.

12. The device of claim 11 wherein said second liquid in said second syringe has a lower ultrasound echogenicity than the liquid in said first container whereby injecting said second liquid locally increases ultrasound visibility.

13. The device of claim 12 wherein said second liquid comprises a pharmaceutical anesthetic.

14. The device of claim 1 wherein said anesthesia needle comprises a local anesthesia needle at least 2.5 centimeters long and having at least one echogenic marker thereon.

15. The device of claim 1 wherein said tubing is at least 40 centimeters long.

16. The device of claim 1 and further comprising at least one marking to indicate the liquid contained therein, wherein said marking comprises a color indicator.

17. The device of claim 16 wherein said color indicator is teal.

18. The device of claim 1 and further comprising a kit comprising an internal-sterile container with at least said tubing, said first syringe, said first valving and said needle within said container.

19. The device of claim 18 and wherein said kit further contains a second syringe.

20. The device of claim 18 and wherein said kit further contains a wrist band, wherein said wristband comprises a color indicator

21. A medical device for providing liquid injectable into a patient from a first liquid container, comprising:
flexible sterile tubing connectable at a proximal end to the first liquid container;
a needle having a cannula at a distal end of said tubing;
a first syringe having a first plunger, said first syringe connected to said tubing between said needle and the first liquid container;
wherein liquid can be drawn from the first liquid container into said first syringe and then injected from said first syringe into the patient via said needle cannula; and,
a second syringe having a second plunger, said second syringe connected to said tubing, wherein a second liquid different from liquid from the first liquid container can be injected from said second syringe into the patient via said cannula.

22. A method for providing liquid injectable into a patient from a first liquid container, comprising the acts of:
connecting sterile tubing at a proximal end to the first liquid container, said first liquid container containing a first liquid comprising a pharmaceutical anesthetic;
retracting a first plunger of a first syringe to draw said first liquid into said first syringe via sterile flexible tubing connecting said first syringe to said first liquid container;
visualizing a local anesthesia needle in the patient via ultrasound imaging;
during said visualization injecting said first liquid from said first syringe through said tubing and through a cannula of said needle at a distal end of said tubing into a local anesthesia site of the patient; and,
blocking said first liquid from flowing from said first syringe back toward the first liquid container with first valving.

23. The method of claim 21, further comprising:
connecting a second fluid container to said sterile tubing, the second fluid container containing a second liquid having lower echogenicity than said pharmaceutical anesthetic,
injecting said second fluid through said tubing and through a cannula of said needle,
visualizing said second fluid via ultrasound imaging.

24. The method of claim 21 wherein said pharmaceutical anesthetic comprises a bupivacaine liposome suspension.

25. A method for providing liquid injectable into a patient from a first liquid container, comprising the acts of:
injecting a second liquid through said tubing, wherein said second liquid has a lower echogenicity than said first liquid.
